(19) United States
(12) Patent Application Publication
(10) Pub. No.: US 2025/0171317 A1
(43) Pub. Date: May 29, 2025

(54) POROUS SILICA PARTICLES AND METHOD FOR PRODUCING SAME

(71) Applicant: JGC CATALYSTS AND CHEMICALS LTD., Kanagawa (JP)

(72) Inventors: MIKI EGAMI, Fukuoka (JP); Takuji MIYAMOTO, Fukuoka (JP); Ryo MURAGUCHI, Fukuoka (JP)

(21) Appl. No.: 18/840,883

(22) PCT Filed: Mar. 31, 2023
(86) PCT No.: PCT/JP2023/013448

§ 371 Date: Aug. 22, 2024

(30) Foreign Application Priority Data
Mar. 31, 2022 (JP) 2022-061033

(51) Int. Cl. C01B 33/18; A61K 8/02; A61K 8/25; A61Q 19/00; C09K 3/14

(52) U.S. Cl. CPC C01B 33/18; A61K 8/0279; A61K 8/25; A61Q 19/00; C09K 3/1409; C01P 2004/01; C01P 2004/61; C01P 2006/11; C01P 2006/14; C01P 2006/16; C01P 2006/22; C01P 2006/80; C01P 2006/90

(57) ABSTRACT

Provided are porous silica particles having balanced appropriate disintegrability and excellent disintegration uniformity, and a method for producing the porous silica particles. The porous silica particles according to the present invention have a network structure, and are elastically deformed to the breaking point when a compressive force increasing at a rate of 0.001450 gf/sec is applied. The ratio “f/d” of the compressive force f (gf) to the displacement amount d (μm) at the breaking point is 0.05 to 0.10. The pore volume of the porous silica particles is 0.4 to 1.3 cm³/g, the mode value of the pore diameter is 2 to 50 nm, the total pore volume of pores having a size within ±25% of the mode value of the pore diameter is 40% or more of the pore volume, the average shape factor is 0.90 to 1.00, the content rate of particles having a shape factor of less than 0.80 is 3.0 number % or less, and the average compressive strength is 9.8 N/mm² to less than 29.4 N/mm².

### TECHNICAL FIELD

The present invention relates to porous silica particles having disintegrability suitable for abrasives, cosmetics, and the like. In particular, the present invention relates to porous particles having a network structure formed by gathering chain particles.

### BACKGROUND ART

In recent years, more precise polishing using porous silica particles as an abrasive has been studied. For example, disintegrable porous silica particles having an average compressive strength (kgf/mm²) of 0.1 or more and less than 1.0 are disclosed in Patent Document 1 (Japanese Patent No. 6631987), and porous silica particles having an average compressive strength of 1 or more and 100 or less (1 to 100) are disclosed in Patent Document 2 (JP-A-2010-064218).

Further, Patent Document 3 (JP-A-02-061406) discloses a method for producing spherical porous silica particles having a sharp particle size distribution.

### SUMMARY OF INVENTION

### Problems to be Solved by Invention

Patent Document 1 aims to obtain particles that have a low average compressive strength and are extremely easily disintegrated, and paragraph 0015 describes that desired disintegrability cannot be obtained when the average compressive strength is 1.0 kgf/mm² or more, and that 0.1 to 0.4 kgf/mm² is more preferable.

In addition, although Patent Document 2 proposes porous particles having an average compressive strength of 1 to 100 kgf/mm², the porous particles are disintegrated without undergoing elastic deformation. Furthermore, particles having an average particle diameter of about 5 to 20 μm suitable for an abrasive have an average compressive strength of 7 kgf/mm² or more (see Examples).

Patent Document 3 describes that when a gel is used, particles having a large pore volume are obtained, and when drying is performed only with a xerogel, the surface activity of the particles is low, so that the interparticle strength during drying is weak, the particles are damaged during drying or classification, and spherical particles cannot be obtained. Therefore, in order to obtain spherical particles, a silica sol is added to a silica gel. Although the average compressive strength of the porous silica particles produced in this manner is not illustrated, it is described that one of the characteristics is that the hardness is high (page 3, column 6, lines 10 to 11), and therefore it is considered that the porous silica particles are designed to be hardly disintegrated. This can also be understood from the facts that in Examples 1 to 3, a silica sol (SI-30, sodium content: about 12000 to 15000 ppm/silica 100%) having a high sodium content was used (the sodium concentration in the porous silica particles was estimated to be 2000 to 3000 ppm) and that the calcination temperature was as high as 600° C.

In Patent Documents 2 and 3, a dispersion liquid containing silica microparticles is pulverized by a sand mill and then spray-dried to produce porous silica particles. Since the particles are granulated with the pulverized particles, minute particles (small pieces) generated at the time of pulverization connect particles to form particles that are hardly disintegrated. In addition, fragments of media beads used in the sand mill may be mixed. Furthermore, the properties of the finally produced porous particles are not stabilized, due to the influence of the concentration of silica microparticles in the dispersion liquid for use in spray drying, the properties of the dispersion liquid, the blending ratio of the sol to the gel, and the like, so that desired particles are hardly obtained. In addition, the uniformity of the internal structure of the particles and the uniformity among the particles are insufficient, and the disintegrability is not uniform.

### Solution to Problems

The porous silica particles of the present invention have a network structure, and when a compressive force increasing at a rate of 0.001450 gf/sec is applied, the particles are elastically deformed to the breaking point, and the ratio “f/d” of the compressive force f (gf) and the displacement amount d (μm) at the breaking point is in the range of 0.05 to 0.10. In the aggregate powder of the porous silica particles, the pore volume is 0.4 to 1.3 cm³/g, the mode value of the pore diameter is 2 to 50 nm, the total pore volume of pores having a size within ±25% of the mode value of the pore diameter is 40% or more of the pore volume, the content rate of particles having a shape factor of less than 0.80 is 3.0 number % or less, and the average compressive strength is 1.0 kgf/mm² or more and less than 3.0 kgf/mm². Such porous silica particles (powder) have balanced appropriate disintegrability.

Further, a method for producing porous silica particles of the present invention includes: a first step of preparing a dispersion liquid in which chain silica particles and binder silica particles are contained at a mass ratio of 40/60 to 70/30 (binder silica particles/chain silica particles); a second step of continuously or intermittently applying a shear force to the dispersion liquid, charging the dispersion liquid having a viscosity of 8 to 100 mPa·s into a spray dryer to granulate spherical silica particles; and a third step of calcining the spherical silica particles to obtain porous silica particles having a network structure with an average compressive strength of 1.0 kgf/mm² or more and less than 3.0 kgf/mm².

### BRIEF DESCRIPTION OF DRAWINGS

FIG. 1 is an electron micrograph of an example of chain silica particles.

FIG. 2 is an electron micrograph illustrating an appearance of porous silica particles.

### DESCRIPTION OF EMBODIMENTS

The present invention relates to porous silica particles having a network structure, and when a compressive force increasing at a rate of 0.001450 gf/sec is applied to the porous silica particles, the particles are elastically deformed to the breaking point. The ratio “f/d” of the compressive force f (gf) and the displaced amount d (μm) at the breaking point is in the range of 0.05 to 0.10 (the ratio “f/d” is an inclination in the elastic deformation region when the displacement amount d (μm) is plotted on the horizontal axis while the compressive force f (gf) is plotted on the vertical axis, and the smaller the ratio, the weaker a force which causes deformation). Such porous silica particles have a uniform internal structure and little variation in disintegrability. The particles are elastically deformed until disintegration, and lumps uniformized in size are formed by the disintegration. Furthermore, the lumps after disintegration also have small variation in strength. In the powder of the porous silica particles having such characteristics, the pore volume is 0.4 to 1.3 cm³/g, the mode value of the pore diameter is 2 to 50 nm, the total pore volume of pores having a size within ±25% of the mode value of the pore diameter is 40% or more of the entire pore volume, the content rate of particles having a shape factor of less than 0.80 is 3.0 number % or less, and the average compressive strength is 1.0 kgf/mm² or more and less than 3.0 kgf/mm². The particles constituting the powder have not only a uniform internal structure but also small variation (individual difference). Therefore, the powder also has little variation in disintegrability. Such porous silica particles have low average compressive strength even with the same pore volume as the porous silica particles disclosed in Patent Documents 2 and 3.

When such porous silica particles are used as an abrasive, the particles are elastically deformed by the load during polishing, and the contact area with a surface to be polished increases. Therefore, the polishing speed is improved. When a load is further applied, the porous silica particles shift from elastic deformation to disintegration, and lumps having a size and strength suitable for polishing are formed. Since these also serve as polishing abrasive grains, it is possible to perform precise polishing and uniform polishing, which hardly causes polishing scratches. When used as a scrubbing material for cosmetics, the porous silica particles sufficiently function as a scrubbing material with good usability.

However, when the pore volume is less than 0.4 cm³/g, the particles become hard, and desired disintegrability cannot be obtained. On the other hand, when it exceeds 1.3 cm³/g, the particles decrease in strength and disintegrate during particle production, so that a desired average shape factor cannot be obtained. The pore volume is preferably 0.5 to 1.0 cm³/g and more preferably 0.5 to 0.9 cm³/g.

In addition, when the mode value of the pore diameter (the mode pore diameter) is less than 2 nm, the particles become hard, and desired disintegrability cannot be obtained. When it exceeds 50 nm, the particles are disintegrated during particle production, and a desired shape factor cannot be obtained. The mode pore diameter is preferably 5 to 45 nm, more preferably 8 to 35 nm, and further preferably 10 to 25 nm.

When the sum of the volumes of pores having a size within +25% of the mode pore diameter (total pore volume) is determined, and this total pore volume is less than 40% of the above-mentioned pore volume (entire pore volume), the particles are non-uniform in terms of the internal structure, and irregular lumps tend to be formed when the particles are disintegrated. Large lumps with irregular sizes are a factor of polishing scratches. This ratio (mode pore volume rate=total pore volume/entire pore volume) is preferably 45% or more, more preferably 50% or more, and further preferably 55% or more.

In addition, the content rate of particles having a shape factor of less than 0.80 needs to be 3.0 number % or less. When this content rate is more than 3.0 number %, stable disintegrability cannot be obtained. Examples of deformed particles having a low shape factor include (a) particles dried and solidified before being formed into a spherical shape, (b) fragmentary particles disintegrated after being granulated into a spherical shape, and (c) coalescing particles formed by coalescence of a plurality of droplets. In the deformed particle, there is a point as a starting point of disintegration. That is, a stress is concentrated and not uniformly transmitted to the particle, and the particle disintegrates starting therefrom. Therefore, the strength is generally lower than that of a spherical particle. Since the particles are stably disintegrated as the content rate of particles having a shape factor of less than 0.80 is lower in this manner, the content rate thereof is preferably 2.0 number % or less and more preferably 1.5 number % or less.

In addition, when the average compressive strength is less than 1.0 kgf/mm², the particles are easily disintegrated during the production process of a grindstone or the like. When it is 3.0 kgf/mm² or more, desired disintegrability cannot be obtained. The average compressive strength is preferably 1.1 kgf/mm² or more and less than 3.0 kgf/mm² and more preferably 1.2 to 2.8 kgf/mm².

The ratio (inclination “f/d”) between the compressive force f (gf) and the displacement amount d (μm) at the breaking point is more preferably 0.06 to 0.10. In the known particles disclosed in Patent Documents 2 and 3, the particles forming the porous particles are excessively strongly fused to each other, and the porous particles are disintegrated almost at the same time as deformation. That is, this inclination exceeded 0.10, and a large force was required until deformation of the particles starts, but the particles of the present invention are deformed by a weaker force because the structures inside the particles are uniform (without bias). Therefore, when used as polishing abrasive grains, the particles of the present invention are deformed by the load during polishing, and the contact area with a surface to be polished increases, so that the polishing speed is improved.

The average shape factor of the powder of the porous silica particles is preferably 0.90 to 1.00. This improves fluidity. Furthermore, even when the direction in which the load is applied is changed, the force is uniformly transmitted to the particle, so that disintegrability is further stabilized. The shape factor affects not only the strength of individual particles but also the strength as an aggregate (powder) of particles. Therefore, the average shape factor is preferably in the above-described range for stable disintegrability. In addition, when the average shape factor is excessively low, defects occur inside when a grindstone or the like is produced, so that good polishing properties are hardly obtained. The average shape factor is preferably 0.91 to 1.00 and more preferably 0.94 to 1.00.

Here, the porous silica particles having an average particle diameter of 5 to 20 μm have high workability and circularity, and thus are suitable for abrasives and cosmetics. When the average particle diameter is less than 5 μm, the fluidity of the powder is lowered, and the workability is deteriorated. In addition, the polishing speed is not sufficient when used as an abrasive, and sufficient scrubbing feeling is hardly obtained when used as a scrubbing material of cosmetics. On the other hand, it is difficult to increase the circularity of particles having an average particle diameter exceeding 20 μm. The average particle diameter is preferably 5 to 15 μm and more preferably 6 to 13 μm.

In this manner, desirable disintegrability can be obtained even with a particle diameter (5 to 20 μm) suitable for abrasives and cosmetics. In Patent Document 2, minute particles generated during pulverization by a sand mill connect particles to form porous silica particles that are hard to be disintegrated. Therefore, in Examples thereof, the average compressive strength is 9 kgf/mm² when the average particle diameter is 10 μm (Example 5), and the average compressive strength is 7 kgf/mm² when the average particle diameter is 5 μm (Example 1). Thus, the average particle diameter (5 to 20 μm) of the present invention does not satisfy the average compressive strength (1.0 kgf/mm² or more and less than 3.0 kgf/mm², i.e., 9.8 N/mm² or more and less than 29.4 N/mm²) of the present invention. Furthermore, it is illustrated that the average compressive strength is 4 kgf/mm² when the average particle diameter is 3.6 μm (Example 4) and that the average compressive strength tends to decrease as the particle diameter decreases. Therefore, the average compressive strength (1.0 kgf/mm² or more and less than 3.0 kgf/mm²) cannot be achieved with the average particle diameter (5 to 20 μm) in the production method of Patent Document 2.

The variation (CV value) in the compressive strength of the porous silica particles is preferably 15 or less. Accordingly, stable disintegrability is obtained. The CV value of the compressive strength is more preferably 13 or less and further preferably 10 or less.

The sodium content rate of the porous silica particles is preferably 100 to 1000 ppm. Usually, sodium causes fusion of particles. Since the fusion of primary particles affects the disintegrability of the particles, and the fusion of secondary particles affects the shape factor, it is desirable that sodium is not contained as much as possible. However, in the porous silica particles of the present invention, it is preferable that sodium is contained within the above-described range in order to obtain particles having a desired average compressive strength and satisfying each requirement. The sodium content rate is more preferably 200 to 900 ppm and further preferably 300 to 800 ppm.

The packed bulk density of the porous silica particles is preferably 0.4 to 1.0 g/ml. As a result, a large amount of the porous silica particles can be blended in a grindstone, so that the particle density of the grindstone is increased, and the polishing performance is improved. In addition, processing and production of a molded body such as a grindstone are facilitated.

Furthermore, the specific surface area of the porous silica particles is preferably 50 to 400 m²/g. When the specific surface area is in this range, it is easy to obtain particles having both easy disintegration and good polishing properties after disintegration. The specific surface area is more preferably 100 to 350 m²/g and further preferably 150 to 300 m²/g.

Furthermore, the porosity of the porous silica particles is preferably 47 to 92%. When the porosity is within this range, good disintegrability is obtained. The porosity is more preferably 52 to 87% and further preferably 52 to 69%.

A method of measuring each characteristic value described above will be described in Examples.

The porous silica particles of the present invention include chain silica particles and binder silica particles. The chain silica particles are appropriately fixed in an intertwined state by the binder function of the binder silica particles. Therefore, a network structure having appropriate strength is formed.

The average particle diameter (average secondary particle diameter d₂) of the chain silica particles is preferably 50 to 500 nm. When the average particle diameter is in this range, the chain silica particles are not densely packed, so that a desired pore volume is obtained. The average secondary particle diameter d₂ is more preferably 50 to 300 nm. Here, the average secondary particle diameter d₂ is an average value of the longest diameters of arbitrary 100 chain silica particles. The longest diameter of each chain silica particle was measured by a scanning electron microscope.

The chain silica particles have a configuration in which primary particles are linked, and the average particle diameter (average primary particle diameter d₁) of the primary particles is preferably 5 to 50 nm. When the average particle diameter is in this range, the porous silica particles include many fine pores formed and have good disintegrability. The average primary particle diameter d₁ is more preferably 5 to 40 nm. The average primary particle diameter d₁ was determined according to the equivalent sphere conversion formula “d=6000/(2.2× SA)”. In the formula, SA is a specific surface area [m²/g] of the chain silica particles obtained by a BET method by nitrogen adsorption, the conversion factor is 6000, and the density of silica is 2.2 g/cm³. The shape of the primary particles may be a true sphere, an ellipsoid, or another shape.

The ratio (d₂/d₁) of the average secondary particle diameter d₂ to the average primary particle diameter d₁ is preferably 1.6 to 100. Within this range, an appropriate three-dimensional network structure is formed, so that good disintegrability is easily obtained. The ratio is more preferably 3 to 70 and further preferably 4 to 40.

The chain silica particles include linear particles in which primary particles are linked so as to extend in a specific direction and non-linear particles in which primary particles are linked so as to extend in a plurality of directions (whether two-dimensional or three-dimensional). In the case of linear particles, the aspect ratio (major axis/minor axis) of the particles is preferably 1.2 or more, more preferably 1.5 or more, and further preferably 1.8 to 10. Here, the aspect ratios of arbitrary 100 particles were measured using an electron micrograph. The arithmetic average value thereof was taken as the average aspect ratio.

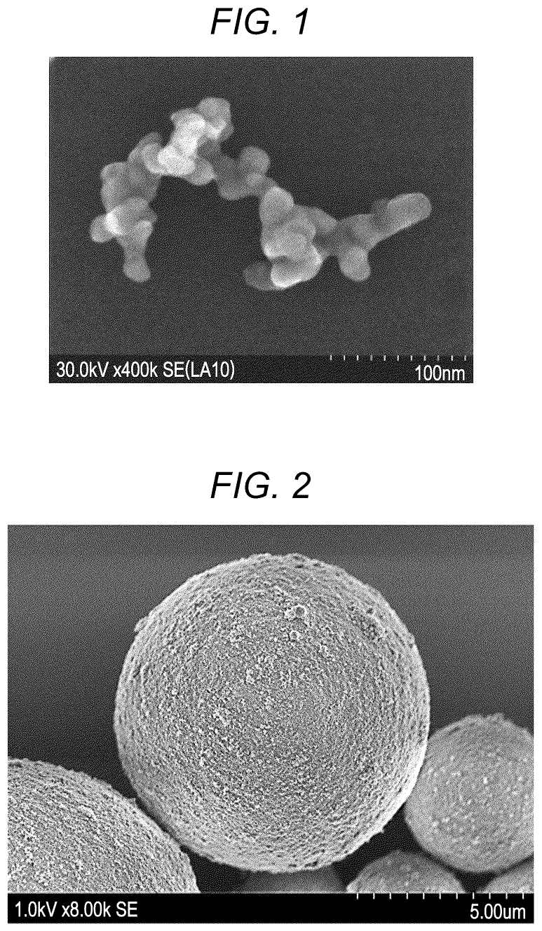

Examples of the non-linear particles include branched particles having a branched structure and bent particles having a bent structure. In practice, there are also particles having both a branched structure and a bent structure. An electron micrograph of an example of such particles is shown in FIG. 1. Here, such particles are also classified as branched particles. When the chain silica particles contain branched particles and bent particles, voids between chain silica particles constituting the porous silica particles are enlarged. Therefore, the porous silica particles are more easily disintegrated. The total content of the branched particles and the bent particles contained in the porous silica particles is preferably 30 mass % or more, more preferably 50 mass % or more, and further preferably 80 mass % or more.

As the chain silica particles, a chain silica sol prepared by a wet method or dry silica (powder) such as fumed silica prepared by a dry method can be used. Specific examples thereof include AEROSIL (registered trademark) manufactured by Nippon Aerosil Co. Ltd., REOLOSIL (registered trademark) manufactured by Tokuyama Corporation, fumed silica for filler usage manufactured by Shin-Etsu Chemical Co., Ltd., fumed silica HDK (registered trademark) manufactured by Asahi Kasei Wacker Silicone Co., Ltd., and silica particles produced by the manufacturing methods described in JP-A-2003-133267 and JP-A-2013-032276.

The binder silica particles used together with the chain silica particles are simple particles (non-chain particles) and have an average particle diameter d that is preferably smaller than the average secondary particle diameter d₂ of the chain silica particles.

The binder silica particles may have a spherical shape (true sphere, ellipsoid) or another shape but are preferably spherical. The aspect ratio is preferably less than 1.2. Further, the average particle diameter is preferably 5 to 100 nm. When the average particle diameter is in this range, a uniform dispersion liquid is obtained when mixed with the chain silica particles. The average particle diameter is more preferably 10 to 50 nm and further preferably 15 to 30 nm.

The average particle diameter of the binder silica particles was calculated from the specific surface area determined by a titration method (the same as the following Sears number).

The binder silica particles preferably contain sodium in order to improve the binder strength. Since sodium causes fusion of silica particles, it is usually desirable that sodium is not contained as much as possible. However, in order to obtain desired binder performance, the sodium content relative to the silica content is preferably 100 to 2000 ppm in the present invention. The sodium content is more preferably 400 to 1600 ppm and further preferably 800 to 1400 ppm.

As the binder silica particles, colloidal silica particles having high surface activity (high SiOH) prepared by a wet method are suitable. The Sears number representing the amount of SiOH groups is preferably 3 to 20. The Sears number is measured by titration of sodium hydroxide as described in the document “Analytical Chemistry 28 (1956), 12, 1981-1983” by Sears. Measurement is performed with 0.1 N NaOH, and the amount required for pH 4 to pH 9 is determined for 1.5 g of silica. Specific examples of the binder silica particles include Cataloid SI series or Cataloid S series manufactured by JGC Catalysts and Chemicals Ltd.

The porous silica particles described above can be used as, for example, an abrasive for polishing an industrial product or the like, or a scrubbing material of cosmetics. When used as an abrasive, the porous silica particles are disintegrated in response to a specific load being applied, so that a substrate (object to be polished) is hardly scratched. In addition, since the average particle diameter of the chain particles after disintegration, the average particle diameter of the primary particles, and the average particle diameter of the binder silica particles are small, fine irregularities on the substrate surface can be polished. In particular, the porous silica particles are suitable for finish dry polishing but can also be used for wet polishing. Specifically, the porous silica particles are suitable for polishing a semiconductor substrate, a substrate for a display, a metal plate, a glass plate, and the like. In actual polishing, the porous silica particles are used in the form of a grindstone molded together with other components. Alternatively, in a powder state or in a slurry state in which the porous silica particles are dispersed in a liquid, polishing is performed using a cloth or a pad.

When used in cosmetics, the porous silica particles exhibit high absorption performance because they have high porosity. Therefore, the porous silica particles can be used as an oil absorbing agent for foundation or a carrier of an active ingredient. Since the particles are easily disintegrated, they are suitable for a scrubbing material.

Next, a method for producing porous silica particles will be described.

First, a dispersion liquid of silica particles in which chain silica particles and binder silica particles are dispersed is prepared (first step). The dispersion liquid is continuously or intermittently applied with a shear force to prepare the viscosity of the dispersion liquid to a range of 8 to 100 mPa·s. The dispersion liquid in this viscosity range is charged into a spray dryer. Porous spherical silica particles are granulated from the chain silica particles and the binder silica particles in the dispersion liquid by the spray dryer (second step). In this way, dried spherical silica particles are obtained. Then, the spherical silica particles are calcined to obtain calcined silica particles (third step).

Here, other steps may be provided between the steps described above. For example, in order to adjust the particle size and particle size distribution according to the purpose, a classification step is preferably provided between granulation and calcination.

Hereinafter, each step will be described in detail.

### [First Step (Dispersion Liquid Preparing Step)]

In this step, a dispersion liquid containing chain silica particles and binder silica particles is prepared. It is preferable to use water as a dispersion medium. In addition, the dispersion liquid preferably contains 5 to 30 mass % of silica particles. Within this concentration range, uniformity of the internal structure of the particles and reduction in variation (individual difference) are realized, and porous silica particles having appropriate disintegrability are stably obtained. In the production methods of Patent Documents 2 and 3, the concentration of the dispersion liquid is high, and particles having appropriate disintegrability cannot be obtained. The concentration is more preferably 10 to 20 mass % and further preferably 10 to 15 mass %. In addition, by controlling the concentration of silica particles, granulation and drying can be efficiently performed. When the concentration is excessively low, granulation is less likely to proceed, and the particle diameter tends to decrease. When the concentration is excessively high, the particle diameter increases, sufficient mechanical strength cannot be obtained, and the particles may be broken at the time of production.

The blending mass ratio of the binder silica particles to the chain silica particles (binder silica particles/chain silica particles) is suitably 40/60 to 70/30. The blending mass ratio is preferably 50/50 to 70/30, more preferably 51/49 to 65/35, and particularly preferably 51/49 to 60/40. At such a blending ratio, the internal structure of the particles is uniformized, and variation in the internal structure is suppressed. Accordingly, porous silica particles having appropriate disintegrability can be stably obtained.

The dispersion liquid of silica particles may contain alcohol such as methanol or ethanol. By containing alcohol, shrinkage during drying can be prevented, and particles having high porosity are obtained.

### [Second Step (Drying Step)]

Next, the dispersion liquid of silica particles obtained in the first step is charged into a spray dryer, and granulated and dried (the resulting particles are referred to as dry silica particles). The viscosity of the dispersion liquid to be charged into the spray dryer needs to be within a certain range (8 to 100 mPa s). The viscosity is preferably as low as possible. By lowering the viscosity of the dispersion liquid, the variation in the diameter of the dry silica particles is reduced. At the same time, a uniform internal structure can be realized, and variation (individual difference) of the internal structure is also reduced. Accordingly, porous silica particles having appropriate disintegrability can be obtained. Furthermore, by maintaining the viscosity within the above-mentioned range, the content rate of particles having a shape factor of less than 0.80 can be reduced. The viscosity range is more preferably 10 to 90 mPa·s and further preferably 10 to 80 mPa·s.

A dispersion liquid containing chain silica particles is known to have non-Newtonian properties. Therefore, the dispersion liquid obtained in the first step has a high viscosity at a low shear rate and a low viscosity at a high shear rate. By applying a shear force to this non-Newtonian dispersion liquid, a high shear rate is applied to lower the viscosity, and spray drying is performed while maintaining the viscosity. That is, a shear force is continuously or intermittently applied to the dispersion liquid to control the viscosity to a range of 8 to 100 mPa·s, and the dispersion liquid in this viscosity range is charged into a spray dryer. Furthermore, it is preferable to reduce the fluctuation in the viscosity of the dispersion liquid. Specifically, the viscosity of the dispersion liquid is preferably controlled to within ±30 mPa·s in a period from the start to the end of charging into the spray dryer. For example, when the viscosity of the dispersion liquid at the start of charging into the spray dryer is 50 mPa·s, the dispersion liquid having a viscosity of 20 to 80 mPa·s is charged into the spray dryer until the spray drying is completed. When the viscosity of the dispersion liquid at the start of charging into the spray dryer is 80 mPa·s, the dispersion liquid of 50 to 100 mPa·s is charged into the spray dryer such that the viscosity does not exceed 100 mPa·s. At this time, a shear force is continuously or intermittently applied such that the viscosity of the dispersion liquid to be charged into the spray dryer falls within the above-mentioned range. The fluctuation range of the viscosity is more preferably within ±25 mPa·s and further preferably within ±20 mPa·s.

Incidentally, in Patent Documents 2 and 3, spray drying is performed by preparing the viscosity of the slurry by continuous pulverization, which does not mean that a shearing force is continuously or intermittently applied. Therefore, even if the viscosity is initially in the above-mentioned range, the viscosity is out of this range over time. Thus, porous silica particles having appropriate disintegrability can be obtained.

Note that the constant viscosity leads to a constant droplet size, with the result that a sharp particle diameter distribution is obtained. Furthermore, the reproducibility of the particle diameter distribution is improved by controlling the fluctuation of the viscosity to within the above-described range. Therefore, porous silica particles having an equivalent particle diameter distribution can be stably produced. In addition, by charging the dispersion liquid into the spray dryer while maintaining the viscosity of the dispersion liquid within a certain range, the dispersion liquid can be prevented from being clogged in a pipe for supplying the dispersion liquid to the spray dryer, the nozzle of the spray dryer, or the like, and the production efficiency is improved.

It is preferable that the chain particles are not pulverized when a shear force is applied to the dispersion liquid. When pulverized, the particles are densely filled with pulverized pieces and the like, and the pore volume of the dry silica particles obtained by granulation decreases. In addition, when pulverized, the specific surface area of the particles contained in the dispersion liquid increases, and the number of hydroxyl groups on the particle surface also increases, so that the bonding between the primary particles is strengthened. From these, the average compressive strength may be 3.0 kgf/mm² or more.

Examples of an apparatus for applying a shear force to the dispersion liquid include a Disperse Mill and a homogenizer. Using these devices, conditions (rotational speed or the like) for applying a shearing force may be set as necessary so that the viscosity of the dispersion liquid is maintained in a specific range such that the chain particles are not pulverized.

The temperature of the dispersion liquid is preferably 5 to 50° C., more preferably 10 to 30° C., and further preferably 15 to 25° C. That is, since the liquid temperature also affects the viscosity, the liquid temperature is preferably maintained within the above-described range.

In addition, it is preferable to dry the dry silica particles to a moisture content rate of 1 to 10%. This can reduce the disintegration of particles due to excessive drying and the generation of deformed particles fused by being calcined while coalescing by moisture. In the drying step, spray drying is performed such that the moisture content rate is within the above-described range, and accordingly the dry silica particles can have a shape closer to a true sphere.

As the spray drying method, a known method such as a rotary disk method, a pressure nozzle method, or a two-fluid nozzle method can be adopted, and a two-fluid nozzle method is particularly preferable. The drying temperature in the drying step is preferably 30 to 150° C. and more preferably 40 to 100° C. in terms of the outlet hot air temperature. By drying in this range, sufficient drying can be obtained, and coalescence and fusion among the dry silica particles in the calcination step can be reduced.

### [Third Step (Calcination Step)]

Calcination is usually performed in an air atmosphere. The calcination temperature is preferably 250 to 800° C., more preferably 300 to 600° C., and further preferably 310 to 410° C. By calcinating in this range, moisture remaining in the porous silica particles is reduced. Therefore, the stability of the quality is improved. In addition, excessive fusion among the chain particles forming the porous silica particles due to high heat can be prevented. That is, it is possible to prevent the strength from becoming excessively high.

### [Classification Step]

A classification step is preferably provided between the second step (drying step) and the third step (calcination step). In the classification step, minute particles and coarse particles are removed. The removal of minute particles and the removal of coarse particles may be performed simultaneously or separately. When performing separately, either the removal of minute particles or the removal of coarse particles may be performed first. In addition, each treatment of the removal of minute particles and the removal of coarse particles may be performed a plurality of times.

Here, the minute particles are particles having an average particle diameter of 1/10 or less of the porous silica particles, and the coarse particles are particles having an average particle diameter of 5 times or more. By removing these particles, the particle diameter and the particle shape are uniformized, molding at the time of processing into a molded body such as a grindstone is easy, excessive clogging with the particles is not caused, and a molded body having appropriate hardness can be obtained.

In this classification step, for the purpose of equalizing the particle size of the powder, particle size classification for dividing the powder according to the particle diameter is performed. An example of the operation of this particle size classification is fluid classification such as dry classification or wet classification. Here, dry classification is preferable.

A classifier used for dry classification can be roughly classified into a gravity classifier, an inertial classifier, and a centrifugal classifier in principle. The use of an inertial classifier or a centrifugal classifier enables more precise classification. Examples of a classifier that is less likely to apply a centrifugal force and can precisely classify even light particles include Elbow-Jet manufactured by Nittetsu Mining Co., Ltd., SG Separator manufactured by Japan Three M Co., Ltd., Aerofine Classifier manufactured by Nisshin Engineering Inc., and Microspin manufactured by Nippon Pneumatic Mfg. Co., Ltd. Among them, Elbow-Jet and Aerofine Classifier are preferable.

Furthermore, a sieving treatment for removing particle lumps is preferably performed at least one of after the drying step and after the calcination step. The particle lumps are particles having a particle diameter of more than 50 μm. The sieving treatment is performed appropriately using a sieve with an aperture (mesh number) capable of removing such particle lumps.

Hereinafter, examples of the present invention will be specifically described.

### Example 1

Into a tank having an internal volume of 150 L, 46.4 L of water and 17 kg of colloidal silica (silica concentration: 20 mass %, average particle diameter: 15 nm) were added, and 5.2 kg of AEROSIL-380 (Nippon Aerosil Co., Ltd.) was gradually added while stirring, so that the mixture was well mixed. As a result, a dispersion liquid of silica particles (silica particle concentration: 12.5 mass %) was obtained. At this time, the viscosity of the dispersion liquid was 200 mPa·s. Note that AEROSIL-380 was chain particles containing branched particles and bent particles, and had an average secondary particle diameter d₂ of 150 nm and an average primary particle diameter di of 7 nm. This dispersion liquid is supplied from the tank to a Disperse Mill (manufactured by Hosokawa Micron Corporation), applied with a shearing force, and then discharged into the tank. The dispersion liquid was thus circulated to adjust the viscosity and fed to an opposed two-fluid nozzle of a spray dryer. At this time, the spray drying conditions were a treatment amount of 60 L/Hr, an air/liquid ratio of 2,100, an air flow rate of Mach 1.1, and a drying atmosphere (temperature: 120° C., humidity: 7.2 vol %). As a result, dry silica particles (moisture content rate: 2%) were obtained. The viscosity of the dispersion liquid was measured at a liquid temperature of 25° C., a rotation speed of 100 rpm, and a measurement time of 60 seconds using a B-type viscometer (TVB-10, Rotor No. 1) manufactured by Toki Sangyo Co., Ltd. The viscosity (40 mPa·s) measured by collecting the dispersion liquid discharged from the Disperse Mill into the tank at the start of charging was defined as the viscosity at the start of charging, and the viscosity (20 mPa s) measured by collecting the dispersion liquid remaining in the tank at the end of charging was defined as the viscosity at the end of charging.

The dry silica particles were allowed to stand at 400° C. for 3 hours to obtain a powder of calcined silica particles (porous silica particles of the present invention). An electron micrograph of this calcined silica particles is shown in FIG. 2.

Physical properties of the calcined silica particles were measured and evaluated as follows. The same procedure was performed in other Examples and Comparative Examples. Table 1 illustrates preparation conditions, and Table 2 illustrates measurement and evaluation results together with the outline of the preparation conditions.

### (1) Average Particle Diameter

The average particle diameter of the binder silica (colloidal silica) particles was calculated from the specific surface area determined by a titration method.

For the porous silica particles, the particle size distribution of the powder was measured using a laser diffraction/scattering particle size distribution measuring instrument (Laser Micron Sizer LMS-3000) manufactured by Seishin Enterprise Co., Ltd., and the average particle diameter of the porous silica particles was obtained by calculating average particle size based on the volume.

### (2) Pore Volume, Mode Pore Diameter and Mode Pore Volume Rate, and Specific Surface Area

Ten grams of a sample (powder of calcined silica particles) was taken in a crucible, and dried at 300° C. for 1 hour. Thereafter, the dried sample was poured in a desiccator and cooled to room temperature. In a glass cell, 0.15 g of the sample was collected. Nitrogen gas was adsorbed to the sample under vacuum degassing using a Belsorp mini II (manufactured by BEL Japan, Inc.) and thereafter desorbed from the sample. The pore volume (V) at a relative pressure of 0.990 was determined from the obtained adsorption isotherm, and the mode pore diameter (peak value) was calculated by a BJH method. The specific surface area was also determined by a BET method.

The mode pore volume rate, which is the ratio of the total pore volume (V_{±25%}) of pores within ±25% of the mode pore diameter to the pore volume (V), is determined by the following equation.

$$Mode pore volume rate ( \% ) = {V}_{\pm 25 \%} / V \times 100$$

### (3) Porosity

The density of silica was set to 2.2 g/cm³ (=0.4545 cm³/g), and the porosity was determined from the pore volume (V) determined by the above-described nitrogen adsorption method according to the following formula.

$$Porosity ( \% ) = V / ( V + 0.4545 ) \times 100$$

### (4) Average Shape Factor and Content Rate of Deformed Particles

Using Morphologi 4 manufactured by Malvern Panalytical Ltd., circularity was measured for about 50,000 particles, and the average value thereof was taken as the average shape factor. The circularity is calculated by a ratio between a circumference of a circle having the same area as a projected object and a perimeter of the object. The ratio at which particles having a circularity of less than 0.8 were present (the number of particles having a circularity of less than 0.8/the number of measured particles) was defined as the content rate of deformed particles.

### (5) Average Compressive Strength and Variation Thereof (CV Value), and Elastic Deformability

Using a micro compression tester (MCT-W500) manufactured by Shimadzu Corporation, a load was applied to one particle (sample) at a constant load speed (0.001450 gf/sec), and displacement was measured until the sample was broken. The compressive strength was calculated from the load (f) when the sample was broken. The compressive strength was determined for 10 particles, and the average value thereof was taken as the average compressive strength.

$$Variation in compressive strength ( CV value ) was obtained according to “ standard deviation of compressive strength / average compressive strength \times 100 ” .$$

For 10 particles, the inclination (f/d) in the elastic deformation region of the particle was obtained from the load (f[gf]) and displacement (d[μm]) when the sample was broken. The elastic deformability was evaluated using the average value of the inclinations.

### (6) Sodium Content Rate

Sulfuric acid and hydrofluoric acid are added to a sample (calcined silica powder), and the sample is heated and dried until white sulfuric acid smoke is generated. After drying, nitric acid and pure water were added, the mixture was heated and dissolved, and pure water was further added to dilute the solution to a certain amount. The sodium concentration in the solution was measured using an atomic absorption apparatus (Z-5300 manufactured by Hitachi, Ltd.). The sodium content was calculated from the sodium concentration in the obtained solution, and the ratio to the mass of the sample (sodium content/sample mass) was taken as the sodium content rate.

### (7) Packed Bulk Density

The powder of the calcined silica was poured in a measuring cylinder and shaken to charge the particles, and the packed bulk density was determined from the mass of the powder and the volume after shaking (the scale of the measuring cylinder was read).

Packed bulk density (g/ml)=mass (g) of particles/volume (ml) after shaking

Further, a polishing grindstone including the powder of calcined silica as abrasive grains was prepared. That is, 100 parts by weight of the powder and 50 parts by weight of rubber particles (NBR cured rubber, average particle diameter: 120 μm) as a matrix were uniformly mixed, and the mixture was compression-molded at a pressure of 100 kgf/cm² into a ring shape. Thereafter, compression heating was performed at 150° C. for 10 minutes to obtain a polishing grindstone having a shape of 300 mm in outer diameter, 100 mm in inner diameter, and 10 mm in thickness. The obtained polishing grindstone was evaluated as follows. The results are illustrated in Table 1.

### [Defects of Grindstone (Yield)]

The appearance of the grindstone was visually evaluated.
Defects are hardly observed: Good
Defects are partially observed: Fair
Many defects are observed, and it cannot be used as a grindstone: Poor

### [Scratch Evaluation]

The polishing grindstone and a base plate were bonded to each other, a flat surface portion of the polishing grindstone was brought into contact with a glass substrate, and the glass substrate was polished under the following polishing conditions. Then, the polished surface of the glass substrate was observed using an ultrafine defect visualization macro apparatus (MICROMAX manufactured by Vision Psytech), and scratches were evaluated according to the following evaluation criteria.

### Polishing Conditions

Grindstone rotation speed: 30 m/sec (peripheral portion)
Grindstone pressurization: 150 g/cm²
Polishing solution: water
Workpiece: glass substrate (borosilicate glass)
Polishing time: 2 minutes 30 seconds

### Evaluation Criteria of Scratches

The surface is smooth and hardly scratched: Good
The surface is smooth but slightly scratched: Fair
The surface lacks smoothness and is scratched: Poor

### [Comprehensive Determination]

The grindstone was comprehensively evaluated from defects of the grindstone and scratch evaluation.
Usable as polishing grindstone: Good
Unusable as polishing grindstone: Poor

### Example 2

Dry silica particles prepared in the same manner as in Example 1 were subjected to a classification treatment to remove minute particles and coarse particles. Otherwise, a powder of calcined silica particles was obtained in the same manner as in Example 1.

### Example 3

Into a tank having an internal volume of 150 L, 43.0 L of water and 21.7 kg of colloidal silica (silica concentration: 20 mass %, average particle diameter: 15 nm) were added, and 4.3 kg of AEROSIL-380 (Nippon Aerosil Co., Ltd.) was gradually added while stirring, so that the mixture was well mixed. As a result, a dispersion liquid of silica particles (silica particle concentration: 12.5 mass %) was obtained. At this time, the viscosity of the dispersion liquid was 180 mPa·s. The dispersion liquid was spray-dried in the same manner as in Example 1 to produce dry silica particles (moisture content rate: 2%). The viscosity of the dispersion liquid at the start of charging was 30 mPa·s, and the viscosity at the end of charging was 20 mPa·s. After minute particles and coarse particles were removed from the dry silica particles, the silica particles were subjected to static calcination at 400° C. for 3 hours to obtain a powder of calcined silica particles.

### Example 4

Cation exchange of 11.3 kg of colloidal silica (Cataloid SI-30 manufactured by JGC Catalysts and Chemicals Ltd., average particle diameter: 10 nm, SiO₂ concentration: 30 mass %) was performed so that the sodium content was about 1/7. The obtained colloidal silica and 14.8 L of water were put into a tank having an internal volume of 150 L. To this tank, 2.3 kg of AEROSIL-130 (manufactured by Nippon Aerosil Co., Ltd.) was gradually added while stirring, so that the mixture was well mixed. As a result, a dispersion liquid of silica particles (silica particle concentration: 20.0 mass %) was obtained. At this time, the viscosity of the dispersion liquid was 200 mPa·s. The dispersion liquid was spray-dried in the same manner as in Example 1 to produce dry silica particles (moisture content: 2 mass %). The viscosity of the dispersion liquid at the start of charging was 60 mPa·s, and the viscosity at the end of charging was 25 mPa·s. After minute particles and coarse particles were removed from the dry silica particles, the silica particles were subjected to static calcination at 400° C. for 3 hours to obtain a powder of calcined silica particles. Note that AEROSIL-130 was chain particles containing branched particles, bent particles, and the like and having an average secondary particle diameter d₂ of 180 nm, and had an average primary particle diameter d₁ of 21 nm.

### Example 5

Into a tank having an internal volume of 150 L, 10.3 L of water and 30 kg of colloidal silica (silica concentration: 20 mass %, average particle diameter: 15 nm) were added, and 2.6 kg of AEROSIL-90G (Nippon Aerosil Co., Ltd.) was gradually added while stirring, so that the mixture was well mixed. As a result, a dispersion liquid of silica particles (silica particle concentration: 20.0 mass %) was obtained. At this time, the viscosity of the dispersion liquid was 180 mPa·s. The dispersion liquid was spray-dried in the same manner as in Example 1 to produce dry silica particles (moisture content rate: 2%). The viscosity of the dispersion liquid at the start of charging was 50 mPa·s, and the viscosity at the end of charging was 25 mPa·s. After minute particles and coarse particles were removed from the dry silica particles, the silica particles were subjected to static calcination at 400° C. for 3 hours to obtain a powder of calcined silica particles. Note that AEROSIL-90G was chain particles containing branched particles, bent particles, and the like and having an average secondary particle diameter d₂ of 200 nm, and had an average primary particle diameter d₁ of 30 nm.

### Comparative Example 1

Into a tank having an internal volume of 150 L, 60 L of water was added, and 8.6 kg of AEROSIL-380 (Nippon Aerosil Co., Ltd.) was gradually added while stirring, so that the mixture was well mixed. As a result, a dispersion liquid of silica particles (silica particle concentration: 12.5 mass %) was obtained. At this time, the viscosity of the dispersion liquid was 200 mPa·s. The dispersion liquid was spray-dried in the same manner as in Example 1 to produce dry silica particles (moisture content rate: 2%). The viscosity of the dispersion liquid at the start of charging was 30 mPa·s, and the viscosity at the end of charging was 15 mPa·s. After coarse particles were removed from the dry silica particles, the dry silica particles were left to stand at 400° C. for 3 hours for calcination. As a result, a powder of calcined silica particles was obtained. Incidentally, the atomic absorption apparatus (detection limit: 5 ppm) used here could not measure the sodium concentration of this comparative example. Therefore, in Table 2, the sodium content rate is described as 0.

### Comparative Example 2

Into a tank having an internal volume of 150 L, 51.7 L of water and 33.3 kg of silica sol (Cataloid SI-30 manufactured by JGC Catalysts and Chemicals Ltd., SiO₂ concentration: 30 mass %, average particle diameter: 10 nm) were put, and 40 kg of AEROSIL-200 (manufactured by Nippon Aerosil Co., Ltd., specific surface area: 200 m²/g, average particle diameter: 14 nm) was gradually added while well stirring, so that the mixture was well mixed. As a result, a dispersion liquid of silica particles (silica particle concentration: 40 mass %) was obtained. At this time, the viscosity of the dispersion liquid was 400 mPa·s. The dispersion liquid was continuously pulverized by a sand mill (Pearl Mill 50STS manufactured by Ashizawa Seisakusho) at such a rate that the retention time was 30 minutes. This dispersion liquid was supplied to an opposed two-fluid nozzle, and spray-dried under the conditions of a treatment liquid amount of 60 L/Hr, a nozzle pressure of 0.38 MPa, a drying atmosphere temperature of 120° C., and a humidity of 7.2 vol %. The viscosity of the dispersion liquid at the start of charging was 26 mPa·s, and the viscosity at the end of charging was 350 mPa·s. This powder was subjected to static calcination at 600° C. for 3 hours to obtain a powder of calcined silica particles. Note that AEROSIL-200 was chain particles containing branched particles, bent particles, and the like and having an average secondary particle diameter d₂ of 170 nm, and had an average primary particle diameter d₁ of 14 nm.

### Comparative Example 3

Into a tank having an internal volume of 150 L, 100 kg of silica sol (Cataloid S-20L manufactured by JGC Catalysts and Chemicals Ltd., average particle diameter: 15 nm, SiO₂ concentration: 20 mass %) was put, and 20 kg of a silica gel powder (AEROSIL-380 manufactured by Nippon Aerosil Co., Ltd., specific surface area: 380 m²/g, average particle diameter: 7 nm) was gradually added thereto, so that the mixture was mixed well. As a result, a dispersion liquid of silica particles (silica particle concentration: 33.3 mass %) was obtained. At this time, the viscosity of the dispersion liquid was 300 mPa·s. The dispersion liquid was continuously pulverized by a sand mill (Pearl Mill 50STS manufactured by Ashizawa Seisakusho) at such a rate that the retention time was 30 minutes. This dispersion liquid was supplied to an opposed two-fluid nozzle, and spray-dried in the same manner as in Comparative Example 2 except that the treatment liquid amount was 5 L/Hr. The viscosity of the dispersion liquid at the start of charging was 26 mPa·s, and the viscosity at the end of charging was 250 mPa·s. This powder was subjected to static calcination at 420° C. for 3 hours to obtain a powder of calcined silica particles.

TABLE 1

| | Production step | | | | | | | | | |
|---|---|---|---|---|---|---|---|---|---|---|
| | Dispersion liquid preparing step | | | | | | | | | |
| | | Binder silica dispersion liquid | | | | | | | | |
| | | | Average | | | | Chain silica particles | | | Silica |
| | | | particle | Dispersion | Silica | Silica | Particle | | | particles |
| | | Product | diameter | liquid mass | concentration | mass A | diameter (nm) | | Mass | concentration |
| | Water | name | (nm) | (kg) | (%) | (kg) | Secondary | | B (kg) | (%) |
| Example 1 | 46.4 | [1] | 15 | 17.0 | 20 | 3.4 | 150 | 7 | 5.2 | 12.5 |
| Example 2 | ↑ | [1] | ↑ | ↑ | ↑ | ↑ | ↑ | ↑ | ↑ | ↑ |
| Example 3 | 43.0 | [1] | 15 | 21.7 | 20 | 4.3 | ↑ | ↑ | 4.3 | ↑ |
| Example 4 | 14.8 | [2] | 10 | 11.3 | 30 | 3.4 | 180 | 21 | 2.3 | 20.0 |
| Example 5 | 10.3 | [1] | 15 | 30 | 20 | 6.0 | 200 | 30 | 2.6 | 20.0 |
| Comparative | 60 | None | — | 0 | — | 0.0 | 150 | 7 | 8.6 | 12.5 |
| Example 1 | | | | | | | | | | |
| Comparative | 51.7 | [3] | 10 | 33.3 | 30 | 10.0 | 170 | 14 | 40 | 40.0 |
| Example 2 | | | | | | | | | | |
| Comparative | 0 | [4] | 15 | 100 | 20 | 20.0 | 150 | 7 | 20 | 33.3 |
| Example 3 | | | | | | | | | | |

| | Production step | | | | | | |
|---|---|---|---|---|---|---|---|
| | Dispersion liquid preparing step | | Drying step | | | | |
| | Particles | Dispersion | | Dispersion liquid | | | |
| | mixing | liquid | | viscosity (mPa · s) | | | |
| | ratio | viscosity | | At start of | At end of | Classification | Calcination |
| | (A/B) | mPa · s | Disperser | spraying | spraying | step | step |
| Example 1 | 40/60 | 200 | (1) | 40 | 20 | None | 400° C., |
| | | | | | | | 3 Hr |
| Example 2 | ↑ | ↑ | (1) | 40 | 20 | Remove | ↑ |
| | | | | | | small/large | |
| Example 3 | 50/50 | 180 | (1) | 30 | 20 | Remove | ↑ |
| | | | | | | small/large | |
| Example 4 | 60/40 | 200 | (1) | 60 | 25 | Remove | ↑ |
| | | | | | | small/large | |
| Example 5 | 70/30 | 180 | (1) | 50 | 25 | Remove | ↑ |
| | | | | | | small/large | |
| Comparative | 0/100 | 200 | (1) | 30 | 15 | Remove | 400° C., |
| Example 1 | | | | | | large | 3 Hr |
| Comparative | 20/80 | 400 | (2) | 26 | 350 | None | 600° C., |
| Example 2 | | | | | | | 3 Hr |
| Comparative | 50/50 | 300 | (2) | 26 | 250 | None | 420° C., |
| Example 3 | | | | | | | 3 Hr |

[1] Colloidal silica
[2] Ion-exchanged SI-30
[3] SI-30
[4] S-20L
(1) Disperse mill
(2) Sand mill

TABLE 2

| | Production step | | | | | | | | | |
|---|---|---|---|---|---|---|---|---|---|---|
| | | | Drying step | | | Physical properties of porous silica particles | | | | |
| | Dispersion liquid preparing step | | Dispersion liquid | | | | | | Mode | |
| | Silica | Particles | viscosity | | | Average | | Mode | pore | Specific |
| | particles | mixing | (mPa · s) | | | particle | Pore | pore | volume | surface |
| | concentration | ratio | At start of | At end of | Classification | diameter | volume | diameter | rate | area |
| | (%) | (A/B) | spraying | spraying | step | μm | cm³/g | nm | % | m²/g |
| Example 1 | 12.5 | 40/60 | 40 | 20 | No | 12 | 0.8 | 18 | 45 | 220 |
| Example 2 | ↑ | ↑ | 40 | 20 | Yes | 9 | 0.8 | 18 | 45 | 220 |
| Example 3 | ↑ | 50/50 | 30 | 20 | Yes | 8 | 0.6 | 16 | 60 | 195 |
| Example 4 | 20.0 | 60/40 | 60 | 25 | Yes | 12 | 0.5 | 14 | 65 | 190 |
| Example 5 | 20.0 | 70/30 | 50 | 25 | Yes | 10 | 0.5 | 12 | 70 | 180 |
| Comparative | 12.5 | 0/100 | 30 | 15 | Yes | 14 | 1.4 | 20 | 43 | 350 |
| Example 1 | | | | | | | | | | |
| Comparative | 40.0 | 20/80 | 26 | 350 | No | 15 | 0.80 | 18 | 30 | 150 |
| Example 2 | | | | | | | | | | |
| Comparative | 33.3 | 50/50 | 26 | 250 | No | 13 | 0.85 | 14 | 35 | 180 |
| Example 3 | | | | | | | | | | |

| | Physical properties of porous silica particles | | | | | | | | | | |
|---|---|---|---|---|---|---|---|---|---|---|---|
| | | | Content | | | | | | | | |
| | | | rate of | | | | | | | | |
| | | | non- | Average | Variation of | Average | Sodium | Packed | | | |
| | | Average | spherical | compressive | compressive | value of | content | bulk | Polishing properties | | |
| | Porosity | shape | particles | strength | strength | inclination | rate | density | Defects of | Scratch | Comprehensive |
| | % | factor | number % | kgf/mm² | CV value | f/d | ppm | g/ml | grindstone | evaluation | determination |
| Example 1 | 64 | 0.95 | 1.0 | 1.2 | 9 | 0.06 | 500 | 0.4 | Good | Good | Good |
| Example 2 | 64 | 0.95 | 1.0 | 1.2 | 9 | 0.06 | 500 | 0.5 | Good | Good | Good |
| Example 3 | 57 | 0.96 | 1.0 | 2.0 | 10 | 0.08 | 625 | 0.5 | Good | Good | Good |
| Example 4 | 52 | 0.96 | 1.0 | 2.8 | 13 | 0.09 | 900 | 0.7 | Good | Good | Good |
| Example 5 | 52 | 0.97 | 1.0 | 2.5 | 12 | 0.09 | 875 | 0.6 | Good | Good | Good |
| Comparative | 76 | 0.90 | 3.0 | 0.37 | 15 | 0.03 | 0 | 0.3 | Fair | Good | Poor |
| Example 1 | | | | | | | | | | | |
| Comparative | 64 | 0.80 | 5.0 | 20.0 | 20 | 0.50 | 2,500 | 0.2 | Good | Poor | Poor |
| Example 2 | | | | | | | | | | | |
| Comparative | 65 | 0.89 | 3.0 | 4.0 | 17 | 0.13 | 1200 | 0.3 | Good | Poor | Poor |
| Example 3 | | | | | | | | | | | |

1. Porous silica particles having a network structure, wherein
when a compressive force increasing at a rate of 0.001450 gf/sec is applied to the porous silica particles, the particles are elastically deformed to a breaking point, and a ratio “f/d” of a compressive force f (gf) and a displacement amount d (μm) at a breaking point is in a range of 0.05 to 0.10, and
a powder of the porous silica particles has following characteristics a) to e),
a) a pore volume is 0.4 to 1.3 cm³/g,
b) a mode value of a pore diameter is 2 to 50 nm,
c) a total pore volume of pores having a size within ±25% of the mode value of a pore diameter is 40% or more of the pore volume,
d) a content rate of particles having a shape factor of less than 0.80 is 3.0 number % or less,
e) an average compressive strength is 1.0 kgf/mm² or more and less than 3.0 kgf/mm².

2. The porous silica particles according to claim 1, wherein the powder has an average particle diameter of 5 to 20 μm.

3. The porous silica particles according to claim 1, wherein the powder has a sodium content rate of 100 to 1000 ppm.

4. The porous silica particles according to claim 1, wherein the powder has a packed bulk density of 0.4 to 1.0 g/ml.

5. An abrasive comprising the porous silica particles according to claim 1.

6. A cosmetic comprising the porous silica particles according to claim 1.

7. A method for producing porous silica particles, comprising:
a first step of preparing a dispersion liquid in which binder silica particles and chain silica particles are contained at a mass ratio of 40/60 to 70/30 (binder silica particles/chain silica particles);
a second step of continuously or intermittently applying a shear force to the dispersion liquid, charging the dispersion liquid having a viscosity of 8 to 100 mPa·s into a spray dryer to granulate spherical silica particles; and
a third step of calcining the spherical silica particles to obtain porous silica particles having a network structure with an average compressive strength of 1.0 kgf/mm² or more and less than 3.0 kgf/mm².

8. The method for producing porous silica particles according to claim 7, wherein the dispersion liquid obtained in the first step has a silica particle concentration of 5 to 30 mass %.
